(19) United States
(12) Patent Application Publication
(10) Pub. No.: US 2017/0212253 A1
(43) Pub. Date: Jul. 27, 2017

(54) ADAPTIVE CT DETECTOR HAVING INTEGRATED READOUT ELECTRONICS

(71) Applicant: General Electric Company, Schenectady, NY (US)

(72) Inventors: Geng Fu, Rexford, NY (US); Peter Michael Edic, Albany, NY (US); Brian David Yanoff, Schenectady, NY (US); Jianjun Guo, Ballston Spa, NY (US); Xin Wang, Clifton Park, NY (US); Bruno Kristiaan Bernard De Man, Clifton Park, NY (US); Xue Rui, Clifton Park, NY (US); Yannan Jin, Schenectady, NY (US)

(21) Appl. No.: 15/004,127

(22) Filed: Jan. 22, 2016

(51) Int. Cl. G01T 1/24; G01T 1/20; A61B 6/03; H01L 27/146

(52) U.S. Cl. CPC G01T 1/247; H01L 27/14658; G01T 1/2018; A61B 6/032

(57) ABSTRACT

A detector panel is described having readout circuitry integrated with the photodetectors, such as in the light imager panel. The detector is useful in high spatial resolution and low-dose or low-signal imaging contexts and may be used in adaptive 2D binning configurations. Adaptive binning of detector elements may be accomplished using control logic and X-ray intensity detector circuitry capable of assessing an incident X-ray intensity and controlling binning of an associated group of detector elements.

### BACKGROUND

The subject matter disclosed herein relates to the fabrication and use of radiation detectors, including X-ray radiation detectors, having integrated electronic readout circuitry and adaptive detector configuration.

Non-invasive imaging technologies allow images of the internal structures or features of a subject (patient, manufactured good, baggage, package, or passenger) to be obtained non-invasively. In particular, such non-invasive imaging technologies rely on various physical principles, such as the differential transmission of X-rays through the target volume or the reflection of acoustic waves, to acquire data and to construct images or otherwise represent the internal features of the subject.

Computed Tomography (CT) scanners operate by projecting fan-shaped or cone-shaped X-ray beams from an X-ray source. The X-ray source emits X-rays at numerous view angle positions about an object being imaged, such as a patient, which attenuates the X-ray beams as they traverse the object. The attenuated beams are detected by a set of detector elements which produce signals representing the intensity of the incident X-ray beams. The signals are processed to produce data representing the line integrals of the linear attenuation coefficients of the object along the X-ray paths. These signals are typically called “projection data” or just “projections”. By using reconstruction techniques, such as filtered backprojection, useful images may be formulated from the projections. The images may in turn be associated to represent a volume or volumetric rendering of a region of interest. In a medical context, pathologies or other structures of interest may then be located or identified from the reconstructed images or rendered volume.

In practice, the transmission path of the analog signal generated at the detector elements to the digitizing readout circuitry may introduce electronic noise. This noise may result in a lower bound in the radiation intensity incident on the detector that is needed to generate images at the desired quality and may otherwise constrain the image quality that can be obtained.

### BRIEF DESCRIPTION

In one implementation, a radiation detector is provided. The radiation detector includes a radiation conversion layer configured to emit optical photons when exposed to X-rays or gamma rays, and a light imager layer positioned proximate to a second surface of the radiation conversion layer opposite the first surface. The light imager layer includes an array of photodetector elements, each configured to generate electrical signals in response to the emitted optical photons that impact respective photodetector elements and readout circuitry located within the light imager panel configured to detect the radiation intensity, adaptively adjust the detector spatial resolution, read out analog signals from proximate photodetector elements, and output digital signals corresponding to optical photon flux incidence at the array of photodetector elements.

In a further implementation, a method for adaptively binning detector elements for an imaging scan without introducing electronic noise penalty is provided. In accordance with this method, at least one scout scan of a subject of interest is acquired. Based upon the at least one scout scan, subject-specific factors or parameters are derived. One or more imaging application-specific factors may also be accessed. Binning control logic of a radiation detector is parameterized based upon one or more of the subject-specific factors and the imaging application-specific factors. Parameterizing the binning control logic includes specifying, for different view angles during the acquisition scan, the binning state of different respective binnable groups of detector elements. The acquisition scan is performed while using the parameterized binning control logic to generate a set of projection data.

In an additional implementation, a method for adaptively binning detector elements during an imaging scan without introducing an electronic noise penalty is provided. In accordance with this method, initial X-ray intensity values detected for each binnable group of detector elements during a projection data acquisition are compared to one or more thresholds. Based upon the comparison, a respective binning state for each binnable group of detector elements at a given view angle is set based upon the initial detected radiation intensity values such that binning states for binnable groups of detector elements change during the image acquisition as the view angle position changes. Projection data is acquired using the binnable groups of detector elements.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a schematic illustration of an embodiment of a Computed Tomography (CT) system configured to acquire projection data of a patient and to process the data in accordance with aspects of the present disclosure;

FIG. 2 depicts a cross-sectional view of components of a prior art radiation detector;

FIG. 3 depicts a cross-sectional view of components of a radiation detector having integrated readout electronics, in accordance with aspects of the present disclosure;

FIG. 4 depicts a top-view of placement of integrated readout electronics within a detector in conjunction with a 2D collimator grid, in accordance with aspects of the present disclosure;

FIG. 5 depicts a top-view of placement of integrated readout electronics within a detector in conjunction with a 1D collimator grid, in accordance with aspects of the present disclosure;

FIG. 6 depicts a top-view of placement of integrated readout electronics within a high-resolution detector in which pixels are divided into sub-pixels, in accordance with aspects of the present disclosure;

FIG. 7 depicts a generalized circuit view of a 2×2 binning configuration, in accordance with aspects of the present disclosure;

FIG. 8 depicts a schematic view of a prior art non-binning readout of a 2×2 array of pixels;

FIG. 9 depicts a schematic view of a prior art 2×2 binning approach using a digital summer;

FIG. 10 depicts a schematic view of a 2×2 binning approach using a common front-end buffer, in accordance with aspects of the present disclosure;

FIG. 11 depicts a generalized circuit view of binning and control circuitry, in accordance with aspects of the present disclosure;

FIG. 12 depicts a generalized circuit view of a charge digitizer, in accordance with aspects of the present disclosure;

FIG. 13 depicts a process flow corresponding to an adaptive mode of detector operation, in accordance with aspects of the present disclosure; and

FIG. 14 depicts a process flow corresponding to an optimal acquisition mode of detector operation, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

While the following discussion is generally provided in the context of medical imaging, it should be appreciated that the present techniques are not limited to such medical contexts. Indeed, the provision of examples and explanations in such a medical context is only to facilitate explanation by providing instances of real-world implementations and applications. However, the present approaches may also be utilized in other contexts, such as the non-destructive inspection of manufactured parts or goods (i.e., quality control or quality review applications), and/or the non-invasive inspection of packages, boxes, luggage, and so forth (i.e., security or screening applications).

The present approaches relate to the fabrication of a radiation detector using tiled silicon wafer and complementary metal-oxide-semiconductors (CMOS) techniques. In particular, fabrication and use of radiation detector such as for use in X-ray and gamma-ray based imaging techniques (including but not limited to Computed Tomography (CT)), X-ray radiography, X-ray tomosynthesis, and positron emission tomography (PET)), is demonstrated where electronic readout circuitry is integrated with the respective photodetector on a single wafer (e.g., c-Si wafer or die) using CMOS techniques. In certain implementations, the integrated readout electronics reduces or minimizes parasitic impedance, thereby reducing associated noise, and correspondingly offers better noise performance in certain applications including low-dose or low-signal applications. The contemplated detector formed with integrated readout electronics will work in either front-illuminated or back-illuminated configurations.

In certain implementations, the low-noise characteristics of the integrated readout electronics may facilitate certain operations or actions when in use, such as grouping (i.e., binning) of pixels during a scanning operation (i.e., in real-time) to allow adaptive detector configuration during the scan operation itself. In one such example, the spatial resolution (i.e., effective pixel size) of all or part of the detector panel may be adaptively controlled (i.e., selected, changed, or adjusted) during a scanning operation, such as by flexibly or adaptively binning grouped pixels or sub-pixels. Other factors that may be adjusted in view of the observed radiation intensity values during an imaging operation include, but are not limited to gain, signal processing parameters (e.g., signal integration time), and selected pixels for readout. Adjustment of such factors during an examination based on measured values may allow for on-line or real-time configuring the detector to receive a certain radiation intensity or flux, readout field of view (FOV), and/or detector operation mode (energy integration or photon counting). Such real-time optimization may configure the detector for acquiring projection data with improved or optimal signal-to-noise ratio (SNR), improved or optimal dynamic range, and/or improved or optimal saturation characteristics. In this manner, a detector with integrated readout electronics may be used adaptively so as to reduce detector noise, provide low-noise binning capabilities, and automatically control detector configuration to maximize or otherwise improve the signal-to-noise or contrast-to-noise ratio and thus image quality for a given subject and imaging purpose. Further, as discussed herein, such adaptive adjustment of spatial resolution (i.e., effective pixel size) may allow non-uniform spatial sampling during an acquisition, such as different projections or regions-of-interest within a given projection being sampled at different spatial resolutions within a single scan.

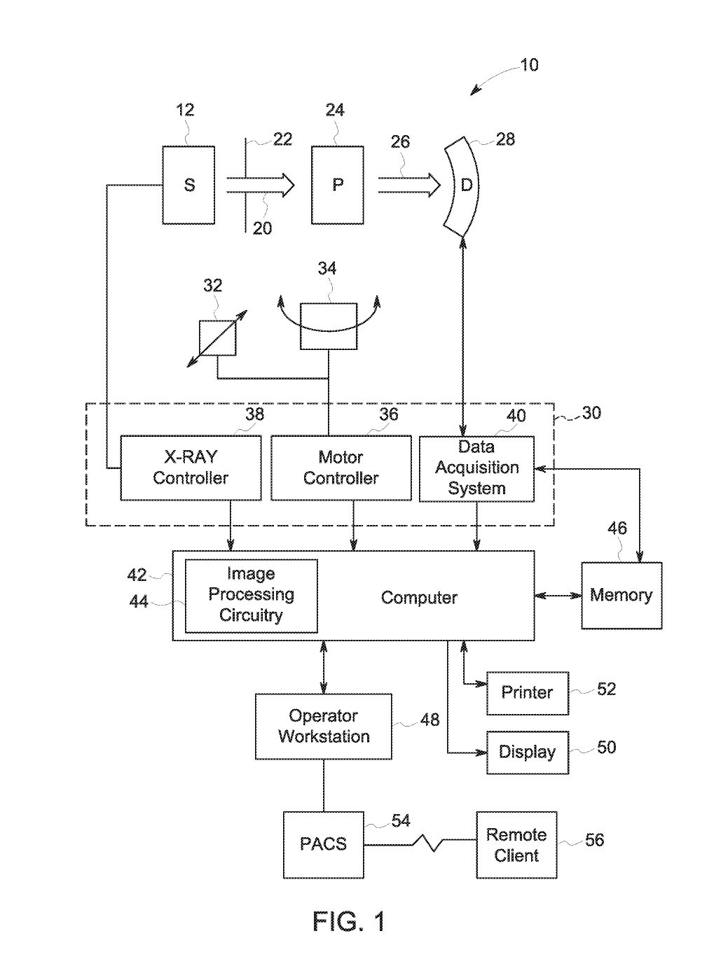

With the preceding discussion in mind, FIG. 1 illustrates an embodiment of an imaging system 10 for acquiring and processing image data in accordance with aspects of the present disclosure. In the illustrated embodiment, system 10 is a Computed Tomography (CT) system designed to acquire X-ray projection data, to reconstruct the projection data into a tomographic image, and to process the image data for display and analysis. As noted above, however, other imaging modalities, both X-ray based and gamma-ray based, may benefit from the present radiation detector design. The depicted CT imaging system 10 includes an X-ray source 12. As discussed in detail herein, the source 12 may include one or more X-ray sources, such as an X-ray tube or one or more enclosures containing solid state emission structures. The X-ray source 12, in accordance with certain contemplated embodiments, is configured to emit an X-ray beam 20 from one or more emission spots (e.g., focal spots), which may correspond to X-ray emission regions on a target structure (e.g., an anode structure) impacted by a directed electron beam.

In certain implementations, the source 12 may be positioned proximate to a filter assembly or beam shaper 22 that may be used to steer the X-ray beam 20, to define the shape and/or extent of a high-intensity region of the X-ray beam 20, to control or define the energy profile of the X-ray beam 20, and/or to otherwise limit X-ray exposure on those portions of the patient 24 not within a region of interest. In practice, the filter assembly or beam shaper 22 may be incorporated within the gantry between the source 12 and the imaged volume.

The X-ray beam 20 passes into a region in which the subject (e.g., a patient 24) or object of interest (e.g., manufactured component, baggage, package, and so forth) is positioned. The subject attenuates at least a portion of the X-rays 20, resulting in attenuated X-rays 26 that impact a detector array 28 formed by a plurality of detector elements (e.g., pixels or sub-pixels) as discussed herein. Each detector element produces an electrical signal that represents the intensity of the X-ray beam incident at the position of the detector element when the beam strikes the detector 28. Electrical signals are acquired and processed to generate one or more scan datasets. In implementations discussed herein, the detector 28 includes integrated readout circuitry and control logic, allowing both the output of digitized signals to downstream components and the adaptive operation of the detector 28, such as adaptive binning of the pixels of the detector 28. In the depicted example, the detector 28 is coupled to the system controller 30, which commands acquisition of the digital signals generated by the detector 28.

A system controller 30 commands operation of the imaging system 10 to execute filtration, examination and/or calibration protocols, and to process the acquired data. With respect to the X-ray source 12, the system controller 30 furnishes power, focal spot location, control signals and so forth, for the X-ray examination sequences. In accordance with certain embodiments, the system controller 30 may control operation of the filter assembly 22, the CT gantry (or other structural support to which the X-ray source 12 and detector 28 are attached), and/or the translation and/or inclination of the patient support over the course of an examination.

In addition, the system controller 30, via a motor controller 36, may control operation of a linear positioning subsystem 32 and/or a rotational subsystem 34 used to move components of the imaging system 10 and/or the subject 24. The system controller 30 may include signal processing circuitry and associated memory circuitry. In such embodiments, the memory circuitry may store programs, routines, and/or encoded algorithms executed by the system controller 30 to operate the imaging system 10, including the X-ray source 12 and/or filter assembly 22, and to process the digital measurements acquired by the detector 28 in accordance with the steps and processes discussed herein. In one embodiment, the system controller 30 may be implemented as all or part of a processor-based system.

The source 12 may be controlled by an X-ray controller 38 contained within the system controller 30. The X-ray controller 38 may be configured to provide power, timing signals, and/or focal size and spot locations to the source 12. In addition, in some embodiments the X-ray controller 38 may be configured to selectively activate the source 12 such that tubes or emitters at different locations within the system 10 may be operated in synchrony with one another or independent of one another or to switch the source between different energy profiles during an imaging session.

The system controller 30 may include a data acquisition system (DAS) 40. The DAS 40 receives data collected by readout electronics of the detector 28, such as digital signals from the detector 28. The DAS 40 may then convert and/or process the data for subsequent processing by a processor-based system, such as a computer 42. In certain implementations discussed herein, circuitry within the detector 28 may convert analog signals of the photodetector to digital signals prior to transmission to the data acquisition system 40. The computer 42 may include or communicate with one or more non-transitory memory devices 46 that can store data processed by the computer 42, data to be processed by the computer 42, or instructions to be executed by a processor 44 of the computer 42. For example, a processor of the computer 42 may execute one or more sets of instructions stored on the memory 46, which may be a memory of the computer 42, a memory of the processor, firmware, or a similar instantiation.

The computer 42 may also be adapted to control features enabled by the system controller 30 (i.e., scanning operations and data acquisition), such as in response to commands and scanning parameters provided by an operator via an operator workstation 48. The system 10 may also include a display 50 coupled to the operator workstation 48 that allows the operator to view relevant system data, imaging parameters, raw imaging data, reconstructed data, contrast agent density maps produced in accordance with the present disclosure, and so forth. Additionally, the system 10 may include a printer 52 coupled to the operator workstation 48 and configured to print any desired measurement results. The display 50 and the printer 52 may also be connected to the computer 42 directly or via the operator workstation 48. Further, the operator workstation 48 may include or be coupled to a picture archiving and communications system (PACS) 54. PACS 54 may be coupled to a remote system 56, radiology department information system (RIS), hospital information system (HIS) or to an internal or external network, so that others at different locations can gain access to the image data.

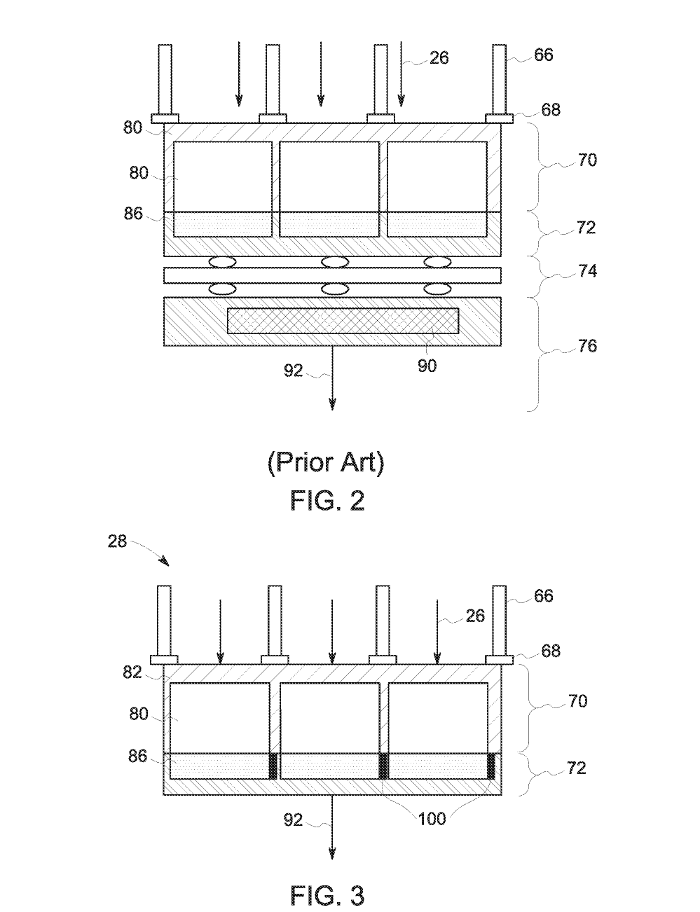

With the preceding discussion of an overall imaging system 10 in mind, a brief discussion of a conventional detector arrangement is provided so as to better distinguish aspects of the present approach. In particular, turning to FIG. 2, a schematic view of a conventional X-ray detector is shown in cross section. The depicted conventional radiation detector of FIG. 2, has certain features in common with, and other features distinct from, the current radiation detector 28.

The depicted detector of FIG. 2 includes a radiation stopping or conversion layer 70, a light imager panel or layer 72 (typically provided as an array of photodetector element, e.g., pixels), a conductive path 74, and readout circuitry 76 configured to readout the light imager panel 72 but provided off-panel with respect to the light imager panel 72. Also depicted are a collimator 66 (e.g., anti-scatter grid) and associated grid plate 68 which may be present to reduce the number of scattered X-ray photons that are incident on the scintillation material 80.

The radiation stopping or conversion layer 70 typically may be provided as a layer of monolithic (i.e., continuous) or pixelated scintillation material 80 that emits lower-energy photons (e.g., optical wavelength photons) when exposed to higher-energy photons 26 (e.g., X-ray or gamma-ray photons). In the depicted example, the scintillation material 80 is pixelated, being separated into individual elements by a light reflecting material 82 that also covers the scintillation material 80 to reduce loss of useful signal.

Low-energy photons emitted by the conversion layer 70 are detected at the light imager panel 72. In particular, the light imager panel 72 typically comprises photodiodes defining an array of photodetector elements (which may correspond to pixels 86 or sub-pixels). Optical photons incident on the photodetector elements result in charge being developed at the respective pixels 86 or sub-pixels which, when read out, corresponds to the incidence of X-rays (or gamma rays) at that location on the detector 28.

The charges at the respective pixels 86 are readout and reset by respective readout circuitry 76 (typically provided as one or more application specific integrated circuits—ASICs 90) that is typically fabricated off-panel with respect to the pixels 86 and which is electrically connected by conductive paths 74 in the form of flex circuitry, bump bonds, or other electrical interconnections. The readout circuitry (e.g., ASICs 90) may include circuitry for amplifer and analog-to-digital conversion (ADC), yielding digital signal 92 that is output from the readout circuitry 76 for subsequent processing.

The detector shown in FIG. 2, while providing useful radiation detection measurements, is notably complex in construction (requiring numerous electrical interconnections between components), with the 3D packaging adding to the cost and degrading performance due to the large interconnect capacitance. Thus, substantial noise is introduced to the measurements due to the length and nature of the electrical interconnect structures and the distance that the analog signal is propagated before digital conversion.

Turning to FIG. 3, in accordance with the present approach a detector 28 is provided that is simpler in design than the conventional detector shown in FIG. 2 and which is less subject to noise. In particular, the detector 28 of FIG. 3 includes integrated readout electronics 100 provided in the light imager panel 72 itself. Such an arrangement simplifies the overall design of the detector 28 and also reduces introduced noise by eliminating the conductive structures 74 (FIG. 1) used to transmit analog signals to the readout and conversion circuitry located off-panel in conventional designs. In this manner, a digital output 92 is output by the light imager panel 72 itself, rather than being generated at an off-panel module. As shown in the depicted example of FIG. 3, the integrated readout electronics 100 is fabricated in the silicon region directly underneath the collimator 66 and grid plate 68, thus shielding the electronics 100 from incident radiation and reducing or eliminating the risk of radiation damage to the electronics 100.

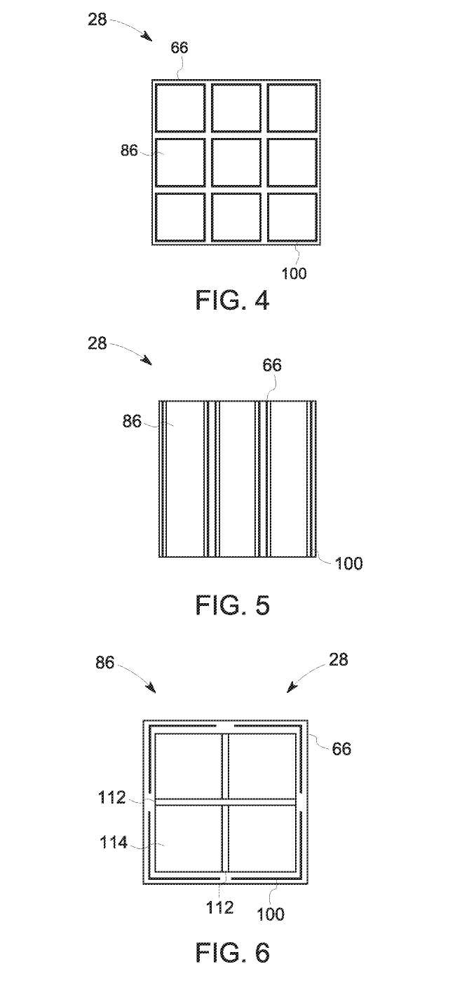

With respect to the placement of the integrated readout electronics 100, various configurations are contemplated. By way of example, and turning to FIGS. 4-6, various configurations are shown that take into account the placement and orientation of a collimator 66 and associated grid plate 68. For example, turning to FIG. 4, a top view is provided of a portion of a detector 28 corresponding to a set of pixels 86. In this top view, the collimator 66 is provided as a two-dimensional (2D) collimator having collimation elements running in vertical and horizontal orientations (in the depicted detector plane), thus forming a grid-like collimator wherein each pixel 86 lies within a cell of the 2D collimation grid. In this example, the integrated readout electronics 100 are provided in a ring or encircling configuration around each pixel 86 corresponding to the placement of the collimator 66. Alternatively, in FIG. 5, the integrated readout electronics 100 are provided in a linear or line configuration corresponding to, and underlying, the one-dimensional (1D) or linear arrangement of the collimator 66 separating linearly arranged pixel regions. Although shown as vertically in FIG. 5, collimator 66 may comprise horizontal features.

An alternative arrangement is provided in FIG. 6, which depicts a high-spatial resolution pixel 86 of a detector 28 in which the pixel 86 is divided into separately addressable sub-pixels 114, each of which has a separate photodiode. In this example, the collimator 66 is provided in a 2D configuration such that a pixel 86 (and corresponding sub-pixels 114) lie within a grid cell of the 2D collimator. In one implementation, the pixel 86 may be approximately 1 mm×1 mm, though the “active area” exposed to radiation may measure about 800 μm×800 μm due to the presence of the overlying collimator 66 and associated grid plate 68. In one such embodiment, each respective sub-pixel 114 may be approximately 400 μm×400 μm, with an insulator (such as air gap or reflector) 112 (≦100 μm across) separating sub-pixels 114 within the pixel 86. In the depicted example, the integrated readout electronics 100 (here sub-pixel readout electronics) are provided along two-edges (i.e., as corner-pieces”) of each sub-pixel 114 such that each sub-pixel 114 has corresponding readout electronics 100 that corresponds to the placement of the collimator 66, though underlying the collimator 66 with respect to incident radiation. As will be appreciated, the sub-pixels 114 in aggregate generally correspond to a single pixel 86 when binned but, when not binned, correspond to separate, individually addressable photodetector elements, each of which separately measures X-ray radiation incidents thereupon. With this in mind, the present binning approaches, discussed below, allow such a high-resolution pixel to be adaptively read out as a single standard resolution pixel without electronic noise penalty when binned or as four high-resolution sub-pixels when read out separately. Although pixel 86 is shown by example as being segmented into 4 sub-pixels in FIG. 6, it is contemplated that pixel 86 could be segmented into alternate configurations, with a smaller or larger number of sub-pixels as required by the given imaging application.

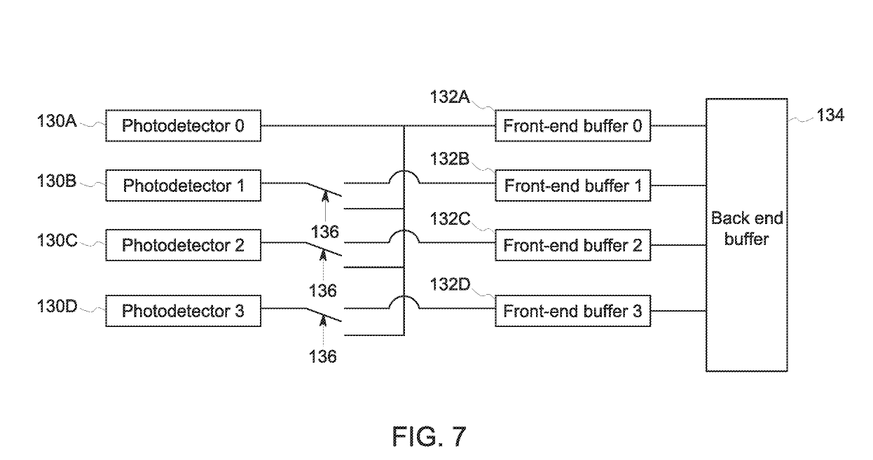

As discussed herein, in certain implementations the detector elements (encompassing both pixels 86 and sub-pixels 114 as used herein) may be adaptively binned during operation using integrated readout electronics 100 so as to obtain all or only a portion of the projection data at higher resolution during a given imaging procedure. By way of example, FIG. 7 depicts a generalized circuit view of a low-noise 2×2 binning arrangement implemented using integrated readout electronics as discussed herein. Though a 2×2 binning arrangement is shown for simplicity, it should be appreciated that other binning arrangements (e.g., 1×2, 2×3, 2×4, 3×3, 3×4, and so forth) may be implemented in accordance with the present approach. Indeed, the present example is provided merely to facilitate explanation and should not be construed as a limiting example.

As will be appreciated from the preceding, though the present examples describe binning a 2×2 arrangement of photodetectors (e.g., detector elements), a given detector may have all of its detector elements defined into such 2×2 (or other) groupings or may have only a portion of its detector elements defined into such groupings, such as a high resolution central region of the detector panel 28. That is, FIG. 7 and the following figures describe the operation and outlay of a single binning group of detector elements, but for a given detector 28 many such binning groups (whether for all or only a portion of the detector elements) may be defined.

In the depicted 2×2 binning example, four photodetectors 130 (e.g., photodiodes) are depicted, such as four pixels 86 in a conventional detector or four sub-pixels 114 within a pixel 86 of a high spatial resolution detector. As noted in the preceding discussion, the photodetectors 130 may be provided in a light detector panel or other layer that includes integrated readout electronics 100 for controlling readout of the photodetectors 130 with low noise. A first photodetector 130A is electrically connected to a first or primary front-end buffer 132A and is always read out to the first front-end buffer 132A. Conversely, the remaining photodetectors 130B, 130C, 130D can be controllably switched (via switches 136), either as a group or separately, between being read out to respective corresponding front-end buffers 132B, 132C, or 132D or to the first front-end buffer 132A. In certain implementations, the front-end buffers 132 are provided as part of the integrated readout electronics 100, and thus are immediately adjacent to the respective photodetectors 130. As a result, the analog signals travel an insubstantial distance from the photodetectors 130 to the front-end buffers 132, thus limiting the introduction of noise.

The front-end buffers 132 output their respective accumulated signals to a shared back-end buffer 134 from which data may be provided to downstream processes. In this manner, each photodetector 130 may be individually read out to separate respective front-end buffers or they may be binned so that one or more of photodetectors 130B, 130C, or 130D is instead read out to the first front-end buffer 132A along with photodetector 130A. In the binning scenario, because the binned photodetectors 130 are read out to the same front-end buffer 132A, this binning operation minimizes the noise impact of the front-end buffer. This is in contrast to conventional binning approaches based on signal multiplexing where each photodetector 130 would output to a separate respective front-end buffer, typically not adjacent to the photodetector, and signal aggregation would occur only at the back-end buffer 134 after the introduction of electronic noise. As will be appreciated, the reduction in noise introduced by the present binning process allows a detector 28 working in accordance with the present approaches to be used effectively at lower doses or at lower signal level and with improved dynamic range compared to conventional processes.

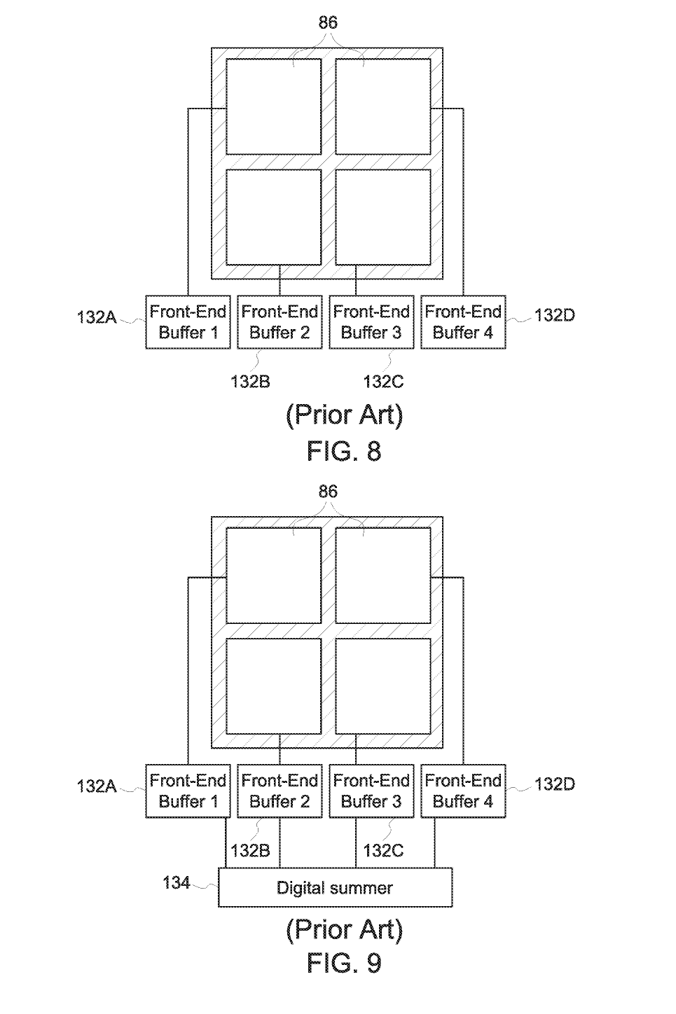

With the preceding in mind, FIGS. 8-10 further illustrate the present low-noise binning approach relative to conventional approaches. Turning to FIG. 8, an example of a conventional independent readout of a 2×2 array of pixels 86 is illustrated. In this example, each of the pixels 86 is read out to a separate respective readout buffer 132. That is, a separate readout to a separate front-end buffer 132 A-D is performed for each pixel 86, with corresponding noise being generated for each readout operation.

Turning to FIG. 9, a conventional binning approach is shown in which a digital summer (e.g., back-end buffer 134) is provided that receives and aggregates (i.e., sums) the output from the respective readout buffers 132 A-D for a 2×2 group of pixels 86. In such an arrangement, a separate readout to a separate front-end buffer 132 is still performed for each pixel 86, though the measured signals are subsequently aggregated at the back-end buffer 134. The SNR may be improved relative to the unbinned example at half (50%) of the dose but with a corresponding loss of spatial resolution. That is the SNR is doubled and the dose cut in half, but spatial resolution is reduced compared to what it is in the example of FIG. 8.

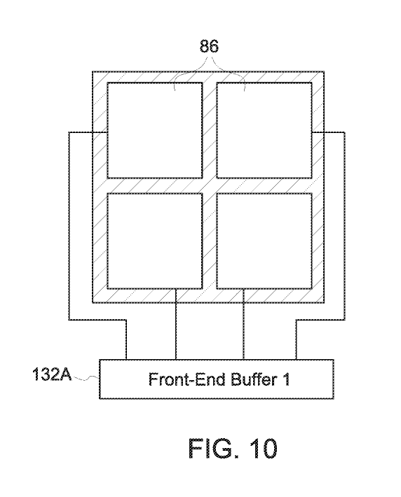

Turning to FIG. 10 a 2×2 binning approach using a detector 28 as discussed herein is shown. In the example, of FIG. 10, the pixels 86 (or sub-pixels 114) are switched to a binning configuration as shown in FIG. 7 (i.e., the switches 136 are all switched so that the pixels 86 output their respective charges to the first front-end buffer 132A, hence binning the pixel charges in the front-end buffer 132A, with a corresponding reduction in electronic noise compared to the conventional binning approaches.

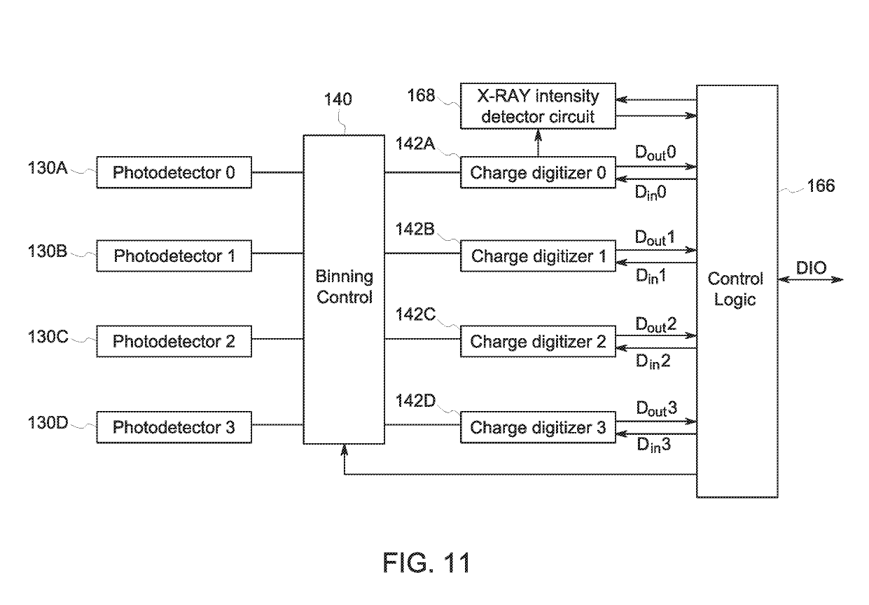

The preceding discussion describes detector features (i.e., low-noise integrated readout circuitry within the light imager panel) and binning approaches consistent with the use of such features. FIG. 11, in turn provides an overview of the binning control and control logic that may be employed to facilitate such binning approaches, such as to allow adaptive binning based on an observed signal-to-noise ratio during an imaging scan. In this example, four photodetectors 130 A-D (e.g., pixels 86 or sub-pixels 114) are shown as being subject to a binning control circuit 140, as discussed above, which controls the states of switches 136 (FIG. 7) and thus whether the photodetectors 130 A-D are binned or not during a given readout operation.

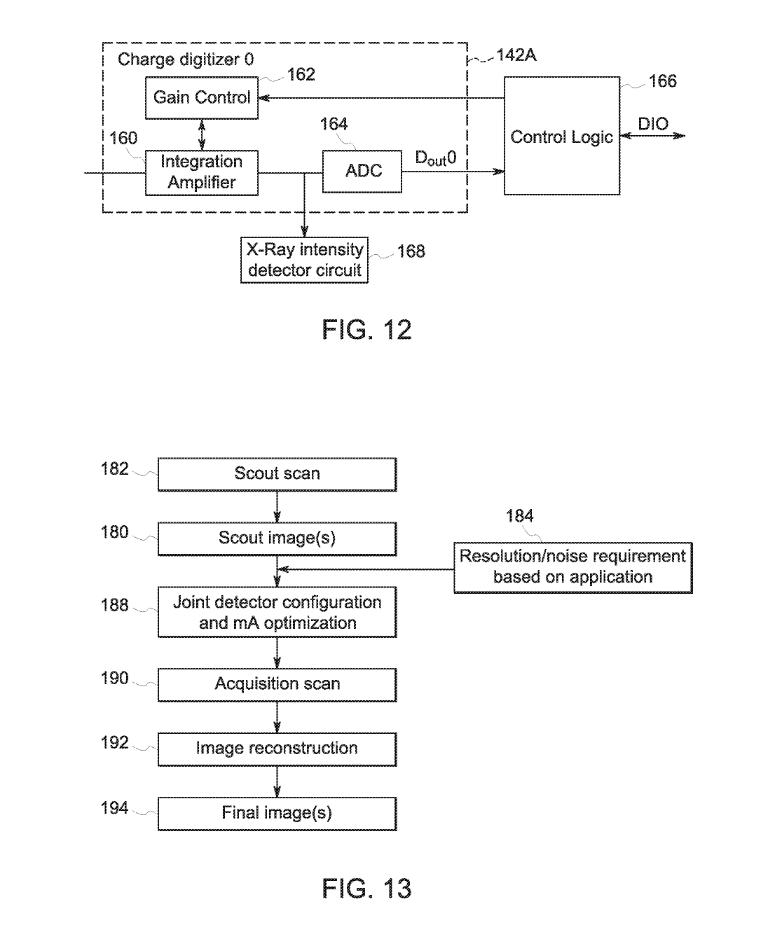

In the depicted example, the binning control circuitry 140 is in communication with charge digitizers 142A, 142B, 142C, 142D, each of which is associated with the integrated readout circuitry corresponding to the respective photodetectors 130 A-D. A schematic view of charge digitizer 142A is provided in FIG. 12 by way of example. As shown in FIG. 12, in one implementation, the charge digitizer 142 includes an integration amplifier 160 in communication with gain control circuitry 162. The amplified signal from the integration amplifier 160 is converted to a digital signal (e.g., Dₒᵤₜ0) at analog-to-digital conversion (ADC) circuitry 164, where it is provided to control logic circuitry 166. Based on the control logic executed by control logic circuitry 166, the gain control 162 may be adjusted so as to control operation of the integration amplifier 160. The control logic circuitry 166 passes the digital signal to downstream processes as a digital input/output signal. In the depicted example, the amplified signal is also output to an X-ray intensity detector circuit 168.

Turning back to FIG. 11, it can be seen that the X-ray intensity detector circuit 168, based on the amplified signal obtained from charge digitizer 142A outputs a control signal or measurement to the control logic circuitry 166. It can also be seen that the control logic circuitry 166 can provide an input to the X-ray intensity detector circuit 168, such as to parameterize the X-ray intensity detector circuit 168 or otherwise establish a threshold or comparison metric for evaluation of the signal obtained from the charge digitizer 142A. In response to the signal or measurement obtained from the X-ray intensity detector circuitry 168, the control logic circuitry 166 may provide instructions to the binning control circuitry 140, such as instructions to bin the outputs of the photodetectors 130 A-D so as to all be readout to front-end buffer 132A (FIG. 7)(in the path corresponding to the charge digitizer 142A) or to not bin the outputs of the photodetectors, allowing each photodetector to be read out to separate respective front end buffers 132 A-D (FIG. 7) and to be separately processed, such as via separate respective charge digitizers 142 A-D.

In this manner, based upon the parameterization and operation of the X-ray intensity detector circuit 168, binning of the photodetectors 130 A-D may be adaptively adjusted (e.g., switched on or off) during a scan operation (i.e., on-the-fly) based on the observed X-ray flux or integrated energy such that some views or regions of interest are imaged at higher resolution than others. Thus, binning may be adaptively adjusted in response to the observed X-ray intensity signal by switching to a binned operation for scan operations (or for views) where the incident radiation on the detector 28 falls below a specified threshold and operating in an unbinned manner when incident radiation is sufficient to support higher resolution operation (i.e., when incident radiation exceeds the specified threshold).

With the flexible or adaptive binning as discussed herein, applications or views that do not require higher resolution may be performed with pixel binning so as to have larger pixel sizes (e.g., larger than the typical pixel size of a CT detector at ˜1 mm×˜1 mm) to improve the signal-to-noise ratio (SNR), thus allowing scanning with lower dose. Such adaptive resolution approaches may be beneficial in certain contexts, such as for ultra-low dose CT scans employed in positron emission tomography (PET) attenuation correction or specific screening applications where resolution is not a key consideration. In such contexts, detector cell binning, as discussed herein, together with sparse view sampling could reduce the CT dose by 3 to 4 times.

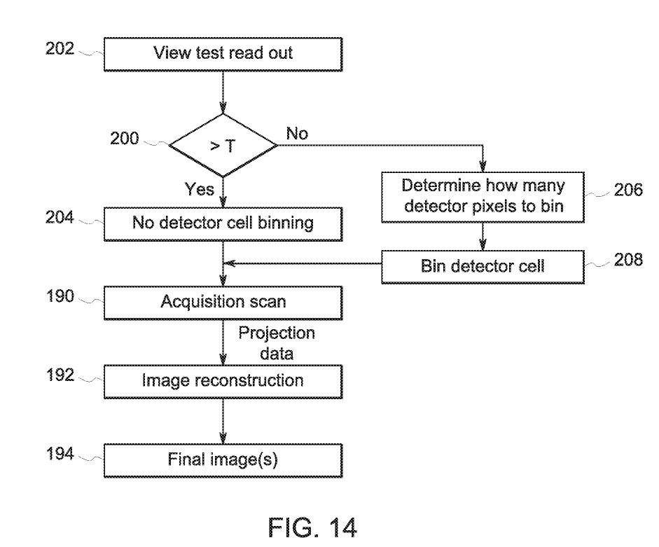

With the preceding discussion in mind, FIGS. 13 and 14 provide examples of process flows that take advantage of the detector optimization (e.g., flexible binning) made possible using the detector 28 and associated integrated readout and control circuitry discussed herein. Turning to FIG. 13, a process flow corresponding to an adaptive mode is illustrated in which the flexible binning capability of the detector 28 is utilized to optimize the detector configuration and X-ray source operation for a given scan. In particular, the depicted process flow is directed toward achieving the lowest dose in combination with an adaptive high-resolution field-of-view (which may correspond to a particular region of interest, such as an organ of interest) and based on the tradeoff between resolution and noise for a given patient habitus and application.

In the process flow of FIG. 13, one or more scout scans are acquired (block 182) using a CT imaging system equipped with the low-noise detector, such as a detector having integrated readout electronics as discussed herein. Such a scout scan 182 may constitute a scan acquired at low dose (e.g., over a short exposure time per projection), and/or at one or limited projection angles or views and typically is acquired in only a small fraction of the available imaging time. The data collected during the scout scans 182 are used to generate scout images 180. Such scout images 180 are typically insufficient for diagnostic or clinical purposes but useful for patient positioning and/or parameterization of a subsequent diagnostic scan. In the present example, the scout image 180 is used to derive useful information about the scan protocol for the subject (subject-specific factors), such as required flux rate, localization of the region of interest, and/or derived image statistics.

Based on the imaging application, the spatial resolution requirements (e.g., minimum or optimum spatial resolution) and noise requirements (e.g., maximum or optimum noise) 184 for the scan protocol are known and may be retrieved from an accessible data store; these are denoted as application-specific factors. These known resolution and noise requirements 184 may be used in conjunction with the information derived from the scout image(s) 180 to parameterize the control logic for detector as well as the operating current for the X-ray source for a given patient and imaging protocol (i.e., clinical or imaging application). Such parameterization may be performed in essentially real-time so as to control the detector configuration during the scan process itself. For example in one embodiment, a forward projection of the of the scout image 180 may be performed in view of the specified resolution and noise requirements 184 to determine a high-resolution field of view for the patient suitable for the prescribed imaging protocol.

In this manner, the control logic may be parameterized or otherwise suitably programmed in real-time or near real-time so as to meet the specified (e.g., minimum or optimum) resolution requirement within the identified high-resolution field of view while not exceeding the specified (e.g., maximum or optimum) noise specification. Thus, for a given patient habitus and imaging application, detector resolution within a specified field of view and radiation dose may be optimized while maintaining acceptable levels of image noise. By way of example, based on the scan protocol for the imaging application and the scout image 180, the control logic is programmed or parameterized so as to adaptively control binning (or no binning) of the grouped sets (e.g., 2×2, 2×3, 3×3, and so forth) of detector elements (i.e., pixels or sub-pixels) of the detector so as to achieve the specified resolution for the identified field of view while achieving an acceptable level of image noise. In such an example, binning for given sets of detector elements on the detector may vary based on view angle, such as based on whether the given field of view or region of interest is being imaged by the set of detector elements at a given view angle.

As noted above, in addition to detector configuration (e.g., binning control logic), the operating current (mA) for the X-ray source may be specified (block 188) so as to provide an appropriate X-ray exposure (i.e., dose) in view of the specified detector binning optimization. Once X-ray source and detector are suitably configured, the acquisition scan 190 may be performed to acquire projection data, which may then be reconstructed (block 192) to generate one or more diagnostic or clinical images 194.

While the preceding example relates to an adaptive mode where detector resolution and noise (and source operating current) are adapted based on known standards and a scout image, other operating modes may be possible using the present low-noise detector technology. For example, turning to FIG. 14, an optimal acquisition process flow suitable for on-the-fly pixel binning (i.e., fast-selected, region-of-interest readout) is depicted. In this mode, a CT detector with integrated readout circuitry and low-noise binning capabilities as discussed herein allows for variable pixel pitch and selective region-of-interest readout, which allows for optimized spatial resolution and/or optimized SNR.

In this mode, threshold values (T values) can be specified (such as in the hardware or programmable circuitry) which can be used to evaluate (decision block 200) image data during an acquisition 202, such as an initial or test read out. For example, signal acquired over a fraction of the view time (e.g., less than or equal to 1/10^{th} of the view time, such as 1/100^{th}, or 1/500^{th}, and so forth) may be used to determine if the observed signal is too low, such as by using the X-ray intensity detector circuitry 168 (FIG. 11) associated with the integrated readout circuitry for respective binnable sets (e.g., 2×2, 2×3, 3×3, and so forth) of pixels or sub-pixels of the detector array. If the specified threshold T is exceeded at block 200 based on the value observed at the respective X-ray intensity detector circuitry 168, the corresponding detector elements (e.g., pixels or sub-pixels) associated with that X-ray intensity detector are not binned (block 204). If, however the threshold T is not exceeded for a set of binnable detector elements, a determination (block 206) is made to bin some or all of the binnable detector elements and the selected elements are binned (block 208) for a given view angle by the control logic. While a single threshold T may be employed to create a binary outcome (i.e., all binnable detector elements binned or not binned), in other scenarios multiple thresholds may be employed that allow different degrees of binning. For example, being below T₁ but above T₂ results in two detector elements being binned, being below T₂ but above T₃ results in three detector elements being binned, being below T₃ results in four detector elements being binned, and so forth.

In this manner, different detector element binning arrangements may be provided for each view for each set of binnable detector elements based on a threshold X-ray intensity evaluation. As will be appreciated in view of the preceding discussion, such control of this optimized binning process may be implemented at the level of the control logic provided on the integrated readout circuitry. In view of the on-the-fly binning determinations, the acquisition scan 190 is completed and projection data acquired, which may be reconstructed (block 192) to generate one or more diagnostic or clinical images 194.

Technical effects of the invention include a detector panel having readout circuitry integrated with the photodetectors, such as in the light imager panel. The detector is useful in low-signal and low-dose imaging contexts and may be used in adaptive binning applications. Adaptive binning of detector elements may be accomplished using control logic and X-ray intensity detector circuitry capable of assessing an incident flux or integrated energy and controlling binning of an associated group of detector elements.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A radiation detector, comprising:
a radiation conversion layer configured to emit optical photons when exposed to X-rays or gamma rays;
a light imager layer positioned proximate to a second surface of the radiation conversion layer opposite the first surface; comprising:
an array of photodetector elements, each configured to generate electrical signals in response to the emitted optical photons that impact respective photodetector elements; and
readout circuitry located within the light imager panel configured to detect the radiation intensity, adaptively adjust the detector spatial resolution, read out analog signals from proximate photodetector elements, and output digital signals corresponding to optical photon flux incidence at the array of photodetector elements.

2. The radiation detector of claim 1, wherein the array of photodetector elements comprises an array of pixels or sub-pixels which each comprise a photodiode and readout circuitry.

3. The radiation detector of claim 1, wherein the radiation conversion layer comprises a pixelated scintillator.

4. The radiation detector of claim 1, wherein the light imager panel is fabricated from a single silicon wafer.

5. The radiation detector of claim 1, further comprising a collimator positioned proximate to a first surface of the radiation conversion layer.

6. The radiation detector of claim 1, wherein the readout circuitry has a configuration comprising at least one of a ring, a linear arrangement, a corner or angled configuration.

7. The radiation detector of claim 1, wherein the array of photodetector elements comprise binnable groups of detector elements, wherein detector elements of each binnable group are read out to a common front-end buffer when in a binned mode but to separate respective front-end buffers when in a non-binned mode.

8. The radiation detector of claim 7, whereon the readout circuitry for each binnable group of detector elements comprises binning control circuitry such that different binnable groups of detector elements can be binned differently from one another during a scan.

9. The radiation detector of claim 7, wherein the readout circuitry for each binnable group of detector elements comprises an X-ray intensity detector circuit for the respective binnable group that outputs a detected X-ray intensity signal used to determine whether one or more detector elements of the respective binnable group are binned.

10. The radiation detector of claim 9, wherein the readout circuitry for each binnable group of detector elements executes control logic that: compares the detected X-ray intensity signal at each respective binnable group to one or more specified values, controls operation of the binning control circuitry for the respective binnable group based upon the comparison, and generates control signals used to control a gain associated with the integration amplifier.

11. The radiation detector of claim 7, wherein the readout circuitry for each binnable group of detector elements comprises a separate respective charge digitizer for each photodetector element of the binnable group, wherein each respective charge digitizer comprises an integration amplifier configured to receive a signal from a respective photodetector element, an analog-to-digital converter configured to receive an amplified output of the integration amplifier, and a gain control configured to control operation of the integration amplifier.

12. The radiation detector of claim 11, wherein at least one charge digitizer of each binnable group of detector elements is configured to output its amplified output to the X-ray intensity detector circuit for the respective binnable group.

13. A method for adaptively binning detector elements for an imaging scan without introducing electronic noise penalty, comprising:
acquiring at least one scout scan of a subject of interest;
based upon the at least one scout scan, deriving subject-specific factors or parameters;
accessing one or more imaging application-specific factors;
parameterizing binning control logic of a radiation detector based upon one or more of the subject-specific factors and the imaging application-specific factors, wherein parameterizing the binning control logic comprises specifying, for different view angles during the acquisition scan, the binning state of different respective binnable groups of detector elements; and
performing the acquisition scan while using the parameterized binning control logic to generate a set of projection data.

14. The method of claim 13, wherein the scout scan is acquired at one or both of low dose or over a limited or sampled projection range in comparison to the acquisition scan.

15. The method of claim 13, wherein the subject-specific factors comprises at least one of required flux rate or a region of interest location.

16. The method of claim 13, wherein the imaging application-specific factors comprise at least one of spatial resolution requirements or noise requirements for the imaging application specific factors.

17. The method of claim 13, further comprising specifying an X-ray source operating current based upon the subject-specific factors and the imaging application-specific factors.

18. A method for adaptively binning detector elements during an imaging scan without introducing an electronic noise penalty, comprising:
comparing initial X-ray intensity values for each binnable group of detector elements during an image acquisition to one or more thresholds;
based upon the comparison, setting a respective binning state for each binnable group of detector elements at a given view angle based upon the initial detected radiation intensity values such that binning states for binnable groups of detector elements change during the image acquisition as the view angle position changes; and
acquiring projection data using the binnable groups of detector elements.

19. The method of claim 18, wherein the initial X-ray intensity values are acquired over 1/10^{th} or less of the view time associated with the image acquisition.

20. The method of claim 18, comprising generating the initial X-ray intensity values using the X-ray intensity detector circuitry associated with each binnable group of detector elements

21. The method of claim 18, wherein the act of comparing the initial X-ray intensity values is performed by control logic executed in integrated readout electronics fabricated on a common silicon wafer or die with the detector elements.
